(19) United States
(12) Patent Application Publication
(10) Pub. No.: US 2008/0318804 A1
(43) Pub. Date: Dec. 25, 2008

(54) Es Cell Mutation Method and System

(75) Inventors: Junji Takeda, Suita-shi (JP); Kosuke Yusa, Suita-shi (JP); Kyoji Horie, Hirakata-shi (JP)

Correspondence Address: SEED INTELLECTUAL PROPERTY LAW GROUP PLLC, 701 FIFTH AVE, SUITE 5400, SEATTLE, WA, 98104, US

(73) Assignees: Osaka University, Suita-shi (JP); Carna Bioscienses, Inc., Kobe-shi (JP)

(21) Appl. No.: 11/628,835

(22) PCT Filed: Jun. 7, 2005
(86) PCT No.: PCT/JP2005/010436

§ 371 Date: Feb. 11, 2008

(30) Foreign Application Priority Data
Jun. 8, 2004 (JP) 2004-170587

(51) Int. Cl. C40B 40/02; C12N 5/06; C12N 15/87; C12N 13/00

(52) U.S. Cl. 506/14; 435/325; 435/455; 435/173.1; 435/441; 435/463

(57) ABSTRACT

This is intended to provide a technique for providing a stem cell having a mutation in both alleles (a pair of alleles). A method for producing a stem cell having a mutation in both chains of alleles which comprises: A) the step of providing a stem cell; B) the step of preventing Blm alleles from functioning in the stem cell; and C) the step of inducing mutation in the stem cell. It is also intended to provide a library of stem cells having a mutation in both chains of alleles wherein stem cells involved in the library have the mutation transferred thereinto over the entire genome.

### TECHNICAL FIELD

The present invention relates to technology for modifying stem cells. More specifically, the present invention relates to a method for causing universal mutagenesis of a stem cell (for example, an embryonic stem cell), system thereof, and stem cells obtained thereby.

### BACKGROUND ART

Stem cells, which have pluripotency, such as embryonic stem cells (hereinafter also called “ES cells”) have been of note, since ES cells can differentiate into various organs or tissues. For example, ‘knock-out’ mice can be generated from ES cells by means of gene targeting, and thus it utility is of note.

In particular, in vitro differentiation of ES cells to many different cell types such as haematopoietic cells, neurons and cardiomyocytes (see, for example, Kyba, M. and Daley, G. Q., Exp. Hematol. 31:994-1006 (2003), Kim, J. H. et al., Nature 418: 50-56 (2002); and Parisi, S. et al., J. Cell Biol. 163: 303-314 (2003)=Non-patent literatures 1-3) has been reported, suggesting the possibility of therapeutic applications (see, for example, Reubinoff, B. E., Pera, M. F., Fong, C. Y., Trouson, A. and Bongso, A., Nature Biotechnol. 18: 399-404 (2000); and Thomson, J. A. et al., Science 282: 1145-1147 (1998)=Non-patent literatures 4-5).

The generation of an ES cell library bearing mutations in both alleles (bi-allelic mutations) have been demanded as it is useful for analyzing the molecular mechanism of differentiation and the pluripotency of ES cells.

[Non-patent literature 1]

Kyba, M. and Daley, G. Q., Exp. Hematol. 31: 994-1006 (2003)

[Non-patent literature 2]

### Kim, J. H. et al., Nature 418: 50-56 (2002)

[Non-patent literature 3]

### Parisi, S. et al., J. Cell Biol. 163:303-314 (2003)

[Non-patent literature 4]

### Reubinoff, B. E., Pera, M. F., Fong, C. Y., Trouson, A. and Bongso, A., Nature Biotechnol. 18: 399-404 (2000)

[Non-patent literature 5]

### Thomson, J. A. et al., Science 282: 1145-1147 (1998)

### DISCLOSURE OF INVENTION

### Problem to be Solved by the Invention

The object of the present invention is to provide technology for providing stem cells having mutations on both alleles (biallelic genes).

### Means for Solving the Problems

The present invention solves the above-mentioned problem by unexpectedly finding that conditional disruption of the Bloom's syndrome gene allows the introduction of mutations over the entire genome of cells such as ES cells.

Accordingly, the present invention provides the following:

In one aspect, the present invention provides a stem cell with a modification incorporated into both strands of the alleles thereof.

In one embodiment, the stem cell is embryonic stem cell.

In one embodiment, the Bloom's syndrome (Blm) gene of the stem cell has been deleted or modified such that the Blm gene does not function.

In one embodiment, the Bloom's syndrome gene comprises the sequence set forth in SEQ ID NO: 1 or a variant thereof.

In another aspect, the present invention provides a library of stem cells with a modification incorporated into both strands of the allele thereof, wherein the stem cells included in the library have incorporated the modification over the entire genome thereof.

In this embodiment, the stem cells are embryonic stem cells.

In this embodiment, the Bloom's syndrome (Blm) gene of the stem cells has been deleted or modified such that the Blm gene does not function.

In this embodiment, the Bloom's syndrome gene comprises the sequence set forth in SEQ ID NO: 1 or a variant thereof.

In another aspect, the present invention provides a method for producing a stem cell with a modification incorporated into both strands of the alleles thereof, the method comprising the steps of: A) providing a stem cell; B) making the Bloom's syndrome gene in the stem cell unfunctional; and C) inducing mutation in the stem cell.

In this embodiment, the Bloom's syndrome gene is processed so as to be transiently dysfunctional.

In this embodiment, the Bloom's syndrome gene is processed so as to be transiently dysfunctional in the presence of an agent.

In this embodiment, the agent is selected from the group consisting of tetracycline, doxycyclin, estrogen derivatives and progesteron derivatives.

In this embodiment, the induction of mutation is selected from the group consisting of exposure to a mutagen, use of a transposon gene, exposure to ultraviolet and exposure to radioactive rays.

In this embodiment, the method further comprises the step of inducing homologous recombination.

In this embodiment, the method further comprises the step of inducing homologous recombination in the 4N phase of the cell, thereby inducing cell division after the induction.

In this embodiment, the stem cell is an embryonic stem cell.

In this embodiment, the embryonic stem cell is a mammalian embryonic stem cell.

In another aspect, the present invention provides a stem cell obtained by a method according to the present invention.

In this embodiment, the inventive stem cell is an embryonic stem cell.

In another aspect, the present invention provides a tissue obtained by a stem cell which is obtained by a method according to the present invention.

In another aspect, the present invention provides a biological organism obtained by a stem cell which is obtained by a method according to the present invention.

In another aspect, the present invention provides use of the Bloom's syndrome gene or a variant thereof for mutation of a stem cell.

In this embodiment of the use of the present invention, the Bloom's syndrome gene is disrupted or modified to be unfunctional in the stem cell. In a specific embodiment, the Bloom's syndrome gene comprises the sequence set forth in SEQ ID NO:1 or a variant thereof.

In another aspect, the present invention provides use of the Bloom's syndrome gene or a variant thereof for manufacturing a composition for mutating a stem cell.

In this embodiment of the use for manufacturing a composition of the present invention, the Bloom's syndrome (Blm) gene has been modified such that the Blm gene does not function. In a specific embodiment, the Bloom's syndrome gene comprises the sequence set forth in SEQ ID NO: 1 or a variant thereof.

### DETAILED DESCRIPTION OF THE INVENTION

The chief limitation of phenotype-based genetic screening in mammalian systems is the diploid nature of the genome. Cells deficient in the Bloom's syndrome gene (Blm) show an increased rate of loss of heterozygosity (LOH) (see, German, J., Dermatol. Clin. 13: 7-18 (1995), Groden, J., Nakamura, Y. and German, J., Proc. Natl. Acad. Sci. USA 87:4315-4319 (1990) and Luo, G. et al., Nature Genet. 26: 424-429 (2000)).

In one specific embodiment, we have used a tetracycline-regulated Blm allele (Blm^{tet}) to introduce bi-allelic mutations across the genome in mouse embryonic stem (ES) cells. Transient loss of Blm expression induces homologous recombination not only between sister chromatids but also between homologous chromosomes. We considered that the phenotype of ES cells bearing bi-allelic mutations would be maintained after withdrawal of the tetracycline analogue doxycycline. Indeed, the combination of N-ethyl-N-nitrosourea (ENU) mutagenesis and transient loss of Blm expression enabled us to generate an ES cell library with genome-wide bi-allelic mutations. The library was evaluated by screening for mutants of glycosylphosphatidylinositol (GPI)-anchor biosynthesis, which involve at least 23 genes distributed throughout the genome. Mutants derived from 12 different genes were obtained and two unknown mutants were simultaneously isolated.

Thus, it is understood that these and other advantages of the present invention will be clear to those skilled in the art upon reading and understanding the following Detailed Description of the Invention in view of the appended drawings.

### EFFECTS OF INVENTION

The present invention provides efficient phenotype-based genetic screening and provides efficient technologies for identifying gene functions in ES cells. The present invention also allows the phenotype-based analysis over the entire genome of ES cells.

### BRIEF DESCRIPTION OF DRAWINGS

FIG. 1 shows generation of conditional Blm alleles in ES cells and elevation of SCE. FIG. 1a depicts the Targeting strategy and resulting Blm alleles. A tet cassette containing the neo or puro gene was inserted upstream of the translational initiation site of Blm to generate Blm^{tetN} or Blm^{tetP}, respectively. After Cre expression, these selection markers were deleted, resulting in Blm^{tet}. B, BamHI; S, SacI. Abbreviations for the tet cassette are described in Bond, C. T. et al., Science 289: 1942-1946 (2000). FIG. 1b depicts the Southern blot analysis of targeted clones. Genomic DNA was digested with BamHI, separated by electrophoresis and hybridized with the radiolabelled probe shown in FIG. 1a. FIG. 1c depicts the long-term analysis of Blm expression in Blm^{tet/tet} ES cells by western blot. Expression of β-actin (Actb) was used as a loading control. FIG. 1d depicts the short-term analysis of Blm expression in Blm^{tet/tet} ES cells by western blot. Expression of b-actin (Actb) was used as a loading control. FIG. 1e depicts the SCE of dox-treated (upper panel) or non-treated (lower panel) Blm^{tet/tet} ES cells.

FIG. 2 shows the high frequency of LOH in Blm-deficient ES cells. FIG. 2a depicts a general scheme of mechanism of LOH. Heterozygosity is represented as A/a. When homologous recombination occurs at the 4N stage between homologous chromosomes, cells bearing LOH (A/A or a/a) appear after cell divisions. FIG. 2b depicts a Luria-Delbruck fluctuation analysis of LOH. Frequency of duplication of the Fasl locus containing the neo gene was examined. c, Simple sequence length polymorphism (SSLP) marker analysis of 28 bi-allelic mutants of the Fasl locus. Open and filled squares indicate heterozygosity and LOH, respectively.

FIG. 3 shows the construction of the mutant ES cell library and the screening strategy of GPI-anchord-efective mutants. FIG. 3a depicts the screening of GPI-anchor-defective mutants from the ES library bearing bi-allelic mutations. FIG. 3b depicts a theoretical prediction of the effectiveness of this screening strategy. From left to right, #1 depicts the number of surviving cells after ENU treatment of 2×10⁸ ES cells, #2 depicts the frequency of X-linked Hprt negative cells measured as a ratio of 6-TG resistant colonies, #3 depicts the mutation rate of LOH as described in FIG. 2b, #4 depicts the number of generations (cell cycles) during dox treatment (number in parentheses shows the total number of cell divisions during dox treatment), #5 depicts the frequency of clones bearing bi-allelic mutation per locus (2.3×10⁻⁴×1/2400×7), and #6 depicts the number of independent clones bearing bi-allelic mutation per locus after dox treatment.

FIG. 4 shows the analysis of GPI-anchor-defective mutants. FIG. 4a depicts the complementation analysis of GPI-anchor-defective mutants. Right, GPI-anchored GFP proteins were expressed on the cell surface of PigA-deficient ES cells only when PigA cDNA was supplied. Left, wild-type ES cells were transfected with GFP-GPI as a positive control. FIG. 4b depicts the chromosomal locations of 23 genes involved in GPI-anchor biosynthesis. Red arrowheads indicate 12 genes of which mutants were obtained in this screening; black arrowheads indicate 11 genes of which mutants were not obtained; asterisk indicates a cDNA that has been cloned but not published. FIG. 4c depicts the chromosome location of mutated genes and the number of mutants obtained. The order of mutated genes in each chromosome is given from centromere to telomere. **Mutants containing the same mutation; ***mutants containing different mutations. Numbers in parentheses indicate the number of mutants bearing the same mutation. FIG. 4d depicts the sequence analysis of mutations in PigH. Homozygous mutations were verified. FIG. 4e depicts the sequence analysis of mutations in GPI8. Homozygous mutations were verified. FIG. 4f depicts the FACS analysis of two novel GPI mutants. Top, ES cells were stained with biotinylated anti-HSA (thin line) and anti-Thy-1 (thick line) antibodies, followed by streptavidin-phycoerythrin. HSA and Thy-1 are GPI-anchored proteins. Broken lines (upper panel) indicate control staining profiles without biotinylated antibodies. Bottom, expression pattern of a non-GPIanchored protein, E-cadherin, in mutants.

### DESCRIPTION OF SEQUENCE LISTING

SEQ ID NO: 1 refers to the nucleic acid sequence of Bloom (Blm) gene;
SEQ ID NO: 2 refers to the amino acid sequence of Bloom (Blm) gene;
SEQ ID NO: 3 refers to the nucleic acid sequence of a cassette of the Blm allele, which is conditionally regulated by Tetracyclin, used in Example 1;
SEQ ID NO: 4 refers to the nucleic acid sequence of a neo gene mutant;
SEQ ID NO: 5 refers to the amino acid sequence of a neo gene mutant.

### BEST MODE FOR CARRYING OUT THE INVENTION

Hereinafter, the present invention will be described. It should be understood throughout the present specification that articles for a singular form (e.g., “a”, “an”, “the”, etc. in English) include the concept of their plurality unless otherwise stated. It should be also understood that the terms as used herein have definitions as typically used in the art unless otherwise stated. Accordingly, unless otherwise defined, all technical and scientific terms used herein shall have the same meaning as that generally understood by those skilled in the art to which the present invention pertains. If there is any inconsistency, the present specification precedes, including definitions.

### DEFINITION OF TERMS

Hereinafter, terms specifically used herein will be defined.

The term “cell” is herein used in its broadest sense in the art, referring to a structural unit of the tissue of a multicellular organism, which is capable of self replicating, has genetic information and a mechanism for expressing it, and is surrounded by a membrane structure which isolates the cell from the outside. Cells used herein may be either naturally-occurring cells or artificially modified cells (e.g., fusion cells, genetically modified cells, etc.). Examples of cell sources include, but are not limited to, a single-cell culture; an embryo, blood, or body tissue of normally-grown transgenic animals; a mixture of cells derived from normally-grown cell lines; and the like.

As used herein the term “4N phase” of a cell refers to a period of time in which the chromosomal number of a cell is duplicate in comparison to the normal state (2n). Such periods of time include but are not limited to: for example, G2 phase of the cellular cycle. Furthermore, it can be determined whether a cell is in the 4N phase or not by staining the chromosome thereof with propidium iodide (PI).

As used herein, the term “stem cell” refers to a cell capable of self replication and pluripotency. Typically, stem cells can regenerate an injured tissue. Stem cells used herein may be, but are not limited to, embryonic stem (ES) cells or tissue stem cells (also called tissular stem cells, tissue-specific stem cells, or somatic stem cell). A stem cell may be an artificially produced cell (e.g., fusion cells, reprogrammed cells, or the like used herein) as long as it has the above-described abilities. Embryonic stem cells are pluripotent cells derived from early embryos. An embryonic stem cell was first established in 1981, which has been applied to the production of knockout mice since 1989. In 1998, a human embryonic stem cell was established, which is currently becoming available for regenerative medicine. Tissue stem cells have a relatively limited level of differentiation, unlike embryonic stem cells. Tissue stem cells are present in the particular place of tissues and have an undifferentiated intracellular structure. Therefore, tissue stem cells have a lower level of pluripotency. Tissue stem cells have a higher nucleus/cytoplasm ratio and have few intracellular organelles. Most tissue stem cells have pluripotency, a long cell cycle, and proliferative ability beyond the life of the individual. As used herein, stem cells may be preferably embryonic stem cells, though tissue stem cells may also be employed, depending on the circumstance.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

As used herein, the term “somatic cell” refers to any cell other than a germ cell, such as an egg, a sperm, or the like, which does not transfer its DNA to the next generation. Typically, somatic cells have limited or no pluripotency.

The origin of a stem cell is categorized into the ectoderm, endoderm, or mesoderm. Stem cells of ectodermal origin are mostly present in the brain, including neural stem cells. Stem cells of mesoderm origin are mostly present in bone marrow, including blood vessel stem cells, hematopoietic stem cells, mesenchymal stem cells, and the like. Stem cells of endodermal origin are mostly present in organs, including liver stem cells, pancreas stem cells, and the like. Somatic cells may be herein derived from any germ layer.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from naturally flanking sequences within an organism from which the nucleic acids are derived (i.e., sequences positioned at the 5′ terminus and the 3′ terminus of the nucleic acids).

As used herein, the term “established” in relation to cells refers to a state in which a particular property (pluripotency) of the cell is maintained and the cell undergoes stable proliferation under culture conditions. Therefore, established stem cells maintain pluripotency. In the present invention, use of an established stem cell is preferable as the step of obtaining a new stem cell from a host can be avoided.

As used herein, “non-embryonic” refers to a state which is not directly derived from early embryo. Accordingly, cells derived from a portion of a body other than early embryo included therein, and in addition thereto, cells obtained by modification of an embryonic stem cells (for example, by genetic modification, fusion or the like), are also within the realm of non-embryonic cells.

As used herein, the term “differentiated cell” refers to a cell having a specialized function and form (e.g., muscle cells, neurons, etc.). Unlike stem cells, differentiated cells have no or little pluripotency. Examples of differentiated cells include epidermial cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

As used herein, the terms “differentiation” or “cell differentiation” refers to the phenomenon where two or more types of cells having qualitative differences in form and/or function occur in a daughter cell population derived from the division of a single cell. Therefore, “differentiation” includes the process during which a population (family tree) of cells, which do not originally have a specific detectable feature, acquire a feature, such as the production of a specific protein, or the like. At present, cell differentiation is generally considered to be the state of a cell in which a specific group of genes in the genome are expressed. Cell differentiation can be identified by searching for intracellular or extracellular agents or conditions which elicit the above-described state of gene expression. Differentiated cells are stable in principle. Particularly, animal cells which have been differentiated once rarely re-differentiate into other types of cells.

As used herein, the term “pluripotency” refers to the nature of a cell, i.e., an ability to differentiate into one or more, preferably two or more, tissues or organs. Therefore, the terms “pluripotent” and “undifferentiated” are herein used interchangeably unless otherwise mentioned. Typically, the pluripotency of a cell is limited during development, and in an adult, cells constituting a tissue or organ rarely differentiate into different cells, that is, the pluripotency is usually lost. Particularly, epithelial cells resist to differentiate into other types of epithelial cells. Such differentiation typically occurs in pathological conditions, and is called metaplasia. However, mesenchymal cells tend to easily undergo metaplasia, i.e., alter to other mesenchymal cells, with a relatively simple stimuli. Therefore, mesenchymal cells have a high level of pluripotency. Embryonic stem cells have pluripotency. Tissue stem cells have pluripotency. As used herein, the ability to differentiate in to all types of cells constituting a living organism, such as a fertilized egg, is called “totipotency”, and the term “pluripotency” may include the concept of totipotency. An example of an in vitro assay for determining whether or not a cell has pluripotency, includes, but is not limited to, culturing under conditions for inducing the formation and differentiation of embryoid bodies. Examples of an in vivo assay for determining the presence or absence of pluripotency, include, but are not limited to, implantation of a cell into an immunodeficient mouse so as to form teratoma, injection of a cell into a blastocyst so as to form a chimeric embryo, implantation of a cell into a tissue of an organism (e.g., injection of a cell into ascites) so as to undergo proliferation, and the like.

As used herein, the term “organ” refers to a morphologically independent structure, localized to a particular portion of an individual organism, in which a certain function is performed. In multicellular organisms (e.g., animals, plants), an organ consists of several tissues spatially arranged in a particular manner, each tissue being composed of a number of cells. An example of such an organ includes an organ relating to the vascular system. In one embodiment, organs targeted by the present invention include, but are not limited to, skin, blood vessels, cornea, kidney, heart, liver, umbilical cord, intestine, nerve, lung, placenta, pancreas, brain, peripheral limbs, retina, and the like. As used herein, cells differentiated from a pluripotent cell of the present invention include, but are not limited to: epidermal cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

As used herein, the term “tissue” refers to an aggregate of cells having substantially the same function and/or form in a multicellular organism. “Tissue” is typically an aggregate of cells of the same origin, but may be an aggregate of cells of different origins as long as the cells have the same function and/or form. Therefore, when stem cells of the present invention are used to regenerate tissue, the tissue may be composed of an aggregate of cells of two or more different origins. Typically, a tissue constitutes a part of an organ. Animal tissues are separated into epithelial tissues, connective tissues, muscular tissues, nervous tissues, and the like, on a morphological, functional, or developmental basis. Plant tissues are roughly separated into meristematic tissues and permanent tissues, according to the developmental stage of the cells constituting the tissue. Alternatively, tissues may be separated into single tissues and composite tissues according to the type of cells constituting the tissue. Thus, tissues are separated into various categories.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a composite of a plurality of polypeptide chains. The term also includes naturally-occurring or artificially modified amino acid polymers. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such oligonucleotides specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond are converted to a peptide-nucleic acid bond, an oligonucleotide derivative in which uracil is substituted with C-5 propynyl uracil, an oligonucleotide derivative in which uracil is substituted with C-5 thiazole uracil, an oligonucleotide derivative in which cytosine is substituted with C-5 propynyl cytosine, an oligonucleotide derivative in which cytosine is substituted with phenoxazine-modified cytosine, an oligonucleotide derivative in which ribose is substituted with 2′-O-propyl ribose, and an oligonucleotide derivative in which ribose is substituted with 2′-methoxyethoxy ribose. Unless otherwise indicated, particular nucleic acid sequences also implicitly encompasses conservatively-modified variants thereof (e.g. degenerate codon substitutions) and complementary sequences as well as sequences explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

As used herein the term “nucleic acid molecule” is also used interchangeably with nucleic acid, oligonucleotide, and polynucleotide, and includes cDNA, mRNA, genomic DNA and the like. As used herein, the nucleic acid and the nucleic acid molecule may be within the concept of the term “gene”.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, the term “Bloom's syndrome (Blm) gene” typically includes the structural gene of Bloom's syndrome (Blm) and the promoter of Bloom's syndrome (Blm). As used herein, “gene” may refer to “polynucleotide”, “oligonucleotide” and “nucleic acid”, and/or “protein”, “polypeptide”, “oligopeptide” and “peptide”. As used herein, “gene product” includes “polynucleotide”, “oligonucleotide” and “nucleic acid” and/or “protein”, “polypeptide”, “oligopeptide” and “peptide”, which are expressed by a gene. Those skilled in the art understand what a gene product is, according to the context.

As used herein, the term “homology” in relation to a sequence (e.g., a nucleic acid sequence, an amino acid sequence, etc.) refers to the level of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, the term “similarity” in relation to a sequence (e.g., a nucleic acid sequence, an amino acid sequence, or the like) refers to the level of identity between two or more sequences when conservative substitution is regarded as positive (identical) in the above-described homology. Therefore, homology and similarity differ from each other in the presence of conservative substitutions. If no conservative substitutions are present, homology and similarity have the same value.

As used herein, the comparison of similarity, identity and homology of an amino acid sequence and a nucleotide sequence is calculated with BLAST, a tool for sequence analysis using default parameters.

As used herein, the term “amino acid” may refer to a naturally-occurring or nonnaturally-occurring amino acid as long as it satisfies the purpose of the present invention. The term “amino acid derivative” or “amino acid analog” refers to an amino acid which is different from a naturally-occurring amino acid and has a function similar to that of the original amino acid. Such amino acid derivatives and amino acid analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of the predetermined amino acid or nucleotide in a polypeptide or polynucleotide as a reference for comparison. Particularly, in the case of enzyme molecules, the term refers to an amino acid which is present at a similar position in an active site and similarly contributes to its catalytic activity. For example, in the case of antisense molecules in a given polynucleotide, the term refers to a similar portion in an ortholog corresponding to a particular portion of the antisense molecule. As used herein, it should be understood that with respect to the amino acids responsible for functions of the Bloom's syndrome gene, amino acids corresponding to the other animals to the murine Bloom's syndrome gene are also responsible for such functions.

As used herein, the term “corresponding” gene refers to a gene in a given species, which has, or is anticipated to have, a function similar to that of a predetermined gene in a species as the reference for comparison. When there are a plurality of genes having such a function, the term refers to a gene having the same evolutionary origin. Therefore, a gene corresponding to a given gene may be an ortholog/a species homolog of the given gene. Therefore, genes corresponding to a mouse Bloom's syndrome gene and the like can be found in other animals. Such a corresponding gene can be identified by techniques well known in the art. Therefore, for example, a corresponding gene in a given animal can be found by searching in the sequence database of the animal (e.g., human, rat) using the sequence of a reference gene (e.g., mouse Bloom's syndrome genes, and the like) as a query sequence.

As used herein, the term “nucleotide” may be naturally-occurring or not. “Nucleotide derivative” or “nucleotide analog” are interchangeably used herein to refer to a derivative or an analog which is different from a naturally occurring nucleotide but has a similar function as that of such a nucleotide. Such a nucleotide derivative and nucleotide analog is well known in the art. Examples of such a nucleotide derivative and nucleotide analog include, for example, but are not limited to phosphorothioate, phosphoramidate, methyl phosphonate, chiral methyl phosphonate, 2-O-methyl ribonucleotide, and peptide-nucleic acid (PNA).

As used herein, the term “fragment” with respect to a polypeptide or polynucleotide refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more amino acids. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification.

As used herein the term “Bloom's syndrome gene”, “Bloom gene”, and “Blm gene” are interchangeably used to refer to a causative gene of syndrome by a genetic disorder related to DNA repair, which is a recessive genetic disease with microcephaly or dwarfism. Bloom Syndrome gene includes but is not limited to:

(A) nucleic acid molecules comprising:

(a) a polynucleotide having a base sequence set forth in SEQ ID NO: 1 or a fragment sequence thereof;

(b) a polynucleotide encoding a polypeptide consisting of an amino acid sequence set forth in SEQ ID NO: 2, or a fragment thereof;

(c) a polynucleotide encoding a variant polypeptide consisting of an amino acid sequence set forth in SEQ ID NO: 2 with at least one mutation selected from at least one amino acid substitution, addition and deletion, or a fragment thereof, which possesses a biological activity;

(d) a polynucleotide being a splice variant or allelic variant of the base sequence set forth in SEQ ID NO: 1, or a fragment thereof;

(e) a polynucleotide encoding a species homolog of a polypeptide consisting of an amino acid sequence set forth in SEQ ID NO: 2, or a fragment thereof;

(f) a polynucleotide which hybridizes to any of polynucleotides (a) through (e) or the complement thereof under stringent conditions, and encoding a polypeptide having a biological activity; or

(g) a polynucleotide having at least 70% identity to any of polynucleotides (a) through (e) or the complement thereof under stringent conditions, and encoding a polypeptide having a biological activity; or

(B) nucleic acid molecules encoding a polypeptide comprising:

(a) a polypeptide encoded by a nucleic acid sequence as set forth in SEQ ID NO: 2 or a fragment thereof;

(b) a polypeptide having the amino acid sequence as set forth in SEQ ID NO: 2 having at least one mutation selected from the group consisting of one or more amino acid substitutions, additions, and deletions, wherein the variant peptide has a biological activity;

(c) a polypeptide encoded by a splice variant or allelic variant of the base sequence as set forth in SEQ ID NO: 1;

(d) a species homolog polypeptide of a polypeptide having the amino acid sequence as set forth in SEQ ID NO: 2; or

(e) a polypeptide consisting of an amino acid sequence having at least 70% identity to any one of the polypeptides of (a) to (d), having a biological activity.

As used herein the term “foreign gene” in a biological organism refers to a non naturally-occurring gene. Such a foreign gene may be a gene which has been modified from a naturally-occurring gene in a biological organism, or a gene which is naturally occurring in another biological organism, such as Bloom gene, or a gene which has been artificially synthesized, or a complex (for example, a fusion) thereof. A biological organism comprising such a foreign gene may express a gene product which is not naturally expressed.

As used herein, the term “expression” of a gene product, such as a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

Accordingly, as used herein, “reduction” of “expression” of a gene, a polynucleotide, a polypeptide or the like refers to when the agent of the present invention is subjected to an action, and the amount of expression is significantly reduced compared to that when the agent is not subjected to an action. Preferably, the reduction of expression includes a reduction of the level of polypeptide expression. As used herein, the “increase” of “expression” of a gene, a polynucleotide, a polypeptide or the like refers to when the agent of the present invention is subjected to an action, resulting in an increase in the amount of expression as compared to when the agent is not subjected to an action. Preferably, the expression increase includes a subsequent increase in the level of the polypeptide expression. As used herein, the term “induction” of “expression” of a gene refers to an increase in the level of expression of the gene by acting an agent on a cell. Accordingly, the induction of expression encompasses the expression of the gene when no expression of the gene had been observed, and the increase in the level of expression of the gene when the level of the expression of the gene had already been observed.

As used herein the term “agent” refers to a compound having properties such as those in which a particular cell survive (or survive in a more potent manner), but other particular cells do not survive (or in a less potent manner). Cells may be selected by the presence and absence or strong and weak survival. Such an agent may include, but is not limited to, for example, tetracyclin, doxycyclin, estrogen derivatives and progensteron derivatives and the like. As used herein, such an agent is preferably a gene whose expression is induced or blocked by the presence of a foreign agent (for example, an antibiotic).

As used herein, term “biological activity” refers to activity processed by an agent (e.g., a polypeptide, a protein, etc.) within an organism, including activities exhibiting various functions (e.g., transcription promoting activity). For example, when a certain agent is an antisense molecule, the biological activity thereof is binding to the nucleic acid of interest, the expression inhibition thereby and the like. For example, when a certain agent is an enzyme, the biological activity thereof includes its enzymatic activity. In another example, when a certain agent is a ligand, the biological activity thereof includes the binding of the ligand to the corresponding receptor thereto. The above-described biological activity can be measured by techniques well-known in the art.

As used herein, the term “antisense (activity)” refers to activity which permits specific suppression or reduction of expression of a target gene. The antisense activity is ordinarily achieved by a nucleic acid sequence having a length of at least 8 contiguous nucleotides, which is complementary to the nucleic acid sequence of a target gene (for example, Blm). Such a nucleic acid sequence preferably has a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. These nucleic acid sequences include nucleic acid sequences having at least 70% homology thereto, more preferably at least 80%, even more preferably at least 90%, and still even more preferably at least 95%. The antisense activity is preferably complementary to a 5′ terminal sequence of the nucleic acid sequence of a target gene. Such an antisense nucleic acid sequence includes the above-described sequences having one or several, or at least one, nucleotide substitutions, additions, and/or deletions.

As used herein, the term “RNAi” is an abbreviation of RNA interference and refers to a phenomenon where an agent for causing RNAi, such as double-stranded RNA (also called dsRNA), is introduced into cells and mRNA homologous thereto is specifically degraded, so that the synthesis of gene products is suppressed, and techniques using the phenomenon. As used herein, RNAi may have the same meaning as that of an agent which causes RNAi.

As used herein, the term “an agent causing RNAi” refers to any agent capable of causing RNAi. As used herein, “an agent causing RNAi of a gene” indicates that the agent causes RNAi relating to the gene and that the effect of RNAi is achieved (e.g., suppression of expression of the gene, and the like). Examples of such an agent causing RNAi include, but are not limited to, sequence having at least about 70% homology with the nucleic acid sequence of a target gene or a sequence hybridizable thereto under stringent conditions and RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g., 2-4 nucleotides in length).

Though not wishing to be bound by any theory, a mechanism which causes RNAi is considered to be defined as follows. When a molecule which causes RNAi, such as dsRNA, is introduced into a cell, an RNaseIII-like nuclease having a helicase domain (called dicer) cleaves the molecule at about 20 base pair intervals from the 3′ terminus in the presence of ATP in the case where the RNA is relatively long (e.g., 40 or more base pairs). As used herein, the term “siRNA” is an abbreviation of short interfering RNA and refers to short double-stranded RNA of 10 or more base pairs which are artificially chemically synthesized or biochemically synthesized, synthesized by an organism, or produced by double-stranded RNA of about 40 or more base pairs being degraded within the organism. siRNA typically has a structure comprising a 5′-phosphate and a 3′-OH, where the 3′ terminus projects by about 2 bases. A specific protein is bound to siRNA to form RISC(RNA-induced-silencing-complex). This complex recognizes and binds to mRNA having the same sequence as that of siRNA and cleaves mRNA at the middle of siRNA due to RNaseIII-like enzymatic activity. It is preferable that the relationship between the sequence of siRNA and the sequence of mRNA to be cleaved as a target is a 100% match. However, base mutations at a site away from the middle of siRNA do not completely remove the cleavage activity by RNAi, leaving partial activity, while base mutations in the middle of siRNA have a large influence and the mRNA cleavage activity by RNAi is considerably lowered. By utilizing such a nature, only mRNA having a mutation can be specifically degraded. Specifically, siRNA in which the mutation is provided in the middle thereof is synthesized and is introduced into a cell. Therefore, in the present invention, siRNA per se, as well as an agent capable of producing siRNA (e.g., representatively dsRNA of about 40 or more base pairs) can be used as an agent capable of eliciting RNAi.

Also, though not wishing to be bound by any theory, apart from the above-described pathway, the antisense strand of siRNA binds to mRNA and siRNA functions as a primer for RNA-dependent RNA polymerase (RdRP), so that dsRNA is synthesized. This dsRNA is a substrate for the dicer again, leading to production of new siRNA. It is intended that such a reaction is amplified. Therefore, in the present invention, siRNA per se, as well as an agent capable of producing siRNA are useful. In fact, in insects and the like, for example, 35 dsRNA molecules can substantially completely degrade 1,000 or more copies of intracellular mRNA, and therefore, it will be understood that siRNA per se, as well as an agent capable of producing siRNA, is useful.

In the present invention, double-stranded RNA having a length of about 20 bases (e.g., representatively about 21 to 23 bases) or less than about 20 bases, called siRNA, can be used. Expression of siRNA in cells can suppress expression of a pathogenic gene targeted by the siRNA. Therefore, siRNA can be used for the treatment, prophylaxis, prognosis, and the like of diseases.

The siRNA of the present invention may be in any form as long as it can elicit RNAi.

In another embodiment, an agent capable of causing RNAi may have a short hairpin structure having a sticky portion at the 3′ terminus (shRNA; short hairpin RNA). As used herein, the term “shRNA” refers to a molecule of about 20 or more base pairs in which a single-stranded RNA partially contains a palindromic base sequence and forms a double-strand structure therein (i.e., a hairpin structure). shRNA can be artificially chemically synthesized. Alternatively, shRNA can be produced by linking sense and antisense strands of a DNA sequence in reverse directions and synthesizing RNA in vitro with T7 RNA polymerase using the DNA as a template. Though not wishing to be bound by any theory, it should be understood that after shRNA is introduced into a cell, the shRNA is degraded in the cell to a length of about 20 bases (e.g., representatively 21, 22, 23 bases), and causes RNAi as with siRNA, leading to the treatment effects of the present invention. It should be understood that such an effect is exhibited in a wide range of organisms, such as insects, plants, animals (including mammals), and the like. Thus, shRNA elicits RNAi as with siRNA and therefore can be used as an effective component of the present invention. shRNA may preferably have a 3′ protruding end. The length of the double-stranded portion is not particularly limited, but is preferably about 10 or more nucleotides, and more preferably about 20 or more nucleotides. Here, the 3′ protruding end may be preferably DNA, more preferably DNA of at least 2 nucleotides in length, and even more preferably DNA of 2-4 nucleotides in length.

The agent capable of causing RNAi used in the present invention may be artificially synthesized (chemically or biochemically) or naturally occurring. There is substantially no difference in terms of the effect of the present invention. A chemically synthesized agent is preferably purified by liquid chromatography or the like.

The agent capable of causing RNAi used in the present invention can be produced in vitro. In this synthesis system, T7 RNA polymerase and T7 promoter are used to synthesize antisense and sense RNAs from template DNA. These RNAs are annealed and thereafter introduced into a cell. In this case, RNAi is caused via the above-described mechanism, thereby achieving the effect of the present invention. Here, for example, the introduction of RNA into cell can be carried out using a calcium phosphate method.

Another example of an agent capable of causing RNAi according to the present invention is a single-stranded nucleic acid hybridizable to mRNA, or all nucleic acid analogs thereof. Such agents are useful for the method and composition of the present invention.

As used herein, “polynucleotides hybridizing under stringent conditions” refers to conditions commonly used and well known in the art. Such polynucleotides can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A (adenine) or T (thymine). As used herein, “hybridizable polynucleotide” refers to a polynucleotide which can hybridize to other polynucleotides under the above-described hybridization conditions. Specifically, the hybridizable polynucleotide includes at least a polynucleotide having a homology of at least 60% to the base sequence of DNA encoding a polypeptide having an amino acid sequence as specifically set forth herein, preferably a polynucleotide having a homology of at least 80%, and more preferably a polynucleotide having a homology of at least 95%.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane or the like or a microarray (microassay) in which genomic DNA is attached to a glass plate under stringent hybridization, PCR and in situ hybridization, and the like.

As used herein, the term “primer” refers to a substance required for the initiation of the reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, or at least 95%. An appropriate sequence as a primer may vary depending on the property of the sequence to be synthesized (amplified). Those skilled in the art can design an appropriate primer depending on the sequence of interest. Such a primer design is well known in the art and may be performed manually or using a computer program (e.g., LASERGENE, Primer Select, DNAStar).

As used herein, the term “agent binding specifically to” a certain nucleic acid molecule or polypeptide refers to an agent which has a level of binding to the nucleic acid molecule or polypeptide equal to or higher than a level of binding to other nucleic acid molecules or polypeptides. Examples of such an agent include, but are not limited to, when a target is a nucleic acid molecule, a nucleic acid molecule having a complementary sequence of a nucleic acid molecule of interest, a polypeptide capable of binding to a nucleic acid sequence of interest (e.g., a transcription agent, etc.), and the like, and when a target is a polypeptide, an antibody, a single chain antibody, either of a pair of a receptor and a ligand, either of a pair of an enzyme and a substrate, and the like.

### Modification of Genes

When the above-described modifications are designed, the hydrophobicity indices of amino acids may be taken into consideration. Hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient.

The hydrophilicity index is also useful for the modification of an amino acid sequence of the present invention. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of the conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such allelic variant ordinarily has a the same or a highly similar sequence to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for the estimation of molecular phylogenetic trees. Usually, orthologs in different species may have a function similar to that of the original species. Therefore, orthologs of the present invention may be useful in the present invention.

As used herein modification of Blm gene to render unfunctional may be achieved by modifying the normal function of the Blm gene by the disruption or reduction thereof, operating the cell not to function the Blm gene, rendering the Blm gene by means of an antisense or RNAi, or processing the cell by means of an agent which transiently dysfunction the gene, or the like.

As used herein the term “mutagenesis” refers to that a mutation is caused to a certain gene, and agents for inducing mutagenesis include but are not limited to: for example, mutagens (for example, N-ethyl-N-nitrosourea (ENU), nitrosoamin derivatives and the like), use of transposon gene, exposure to UV or radiation, and the like. It should be understood that those skilled in the art may practice mutagenesis using a transposon in view of the inventors' technology (see, WO 02/13602).

As used herein, the term “conservative (or conservatively modified) variant” applies to both amino acid and nucleic acid sequences. With respect to particular nucleic acid sequences, conservatively modified variants refer to nucleic acids encoding identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variations. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of methods for such modifications of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500 (1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to the original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modifications include, but are not limited to, amidation, carboxylation, sulfation, halogenation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using a technique well known in the art. Therefore, a peptide analog may be a polymer containing an amino acid analog.

Similarly, the term “polynucleotide analog” or “nucleic acid analog” refers to a compound which is different from the polynucleotide or nucleic acid but has at least one chemical function or biological function equivalent to that of the polynucleotide or nucleic acid. Therefore, polynucleotide analogs or nucleic acid analogs include one that has at least one nucleotide analog or nucleotide derivative addition or substitution with respect to the original peptide.

Nucleic acid molecules as used herein may include those in which a part of the sequence of the nucleic acid molecule is deleted or is substituted with other base(s), or an additional nucleic acid sequence is inserted, as long as the polypeptide expressed by the nucleic acid molecule has substantially the same activity as that of the naturally-occurring polypeptide, as described above. Alternatively, additional nucleic acids may be linked to the 5′ terminus and/or 3′ terminus of the nucleic acid molecule. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid molecule can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide sequence. This is achieved by techniques well known in the art, including a site-specific mutagenesis technique and the like. A polypeptide or a polynucleotide may have any number (>0) of substitutions, additions, or deletions. The number can be as large as the variant having such a number of substitutions, additions or deletions maintains the intended function (e.g., the information transfer function of hormones and cytokines, etc.). For example, such a number may be one or several, and preferably within 20% or 10% of the full length, or no more than 100, no more than 50, no more than 25, or the like.

As used herein, the term “specifically expressed” in the case of genes indicates that a gene is expressed in a specific site or in a specific period of time at a level different from (preferably higher than) that in other sites or periods of time. The term “specifically expressed” includes that a gene may be expressed only in a given site (specific site) or may be expressed in other sites. Preferably, the term “specifically expressed” indicates that a gene is expressed only in a given site.

As used herein the term “homologous recombination” refers to a crossover phenomenon in the DNA portions corresponding to each other between chromosomes. For example, such a homologous recombination is achieved by the deletion of Blm gene and using Cre/lox P system. In particular, when a homologous recombination is desired for inducing the 4N phase of the cell, it is achieved by subjection to the conditions of deletion of Blm gene and the use of Cre/loxP system.

Molecular biological, biochemical, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Experimental Method for Gene Introduction & Expression Analysis”, Yodo-sha, 1997; and the like. Relevant portions (or possibly the entirety) of each of these publication are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

When a gene is mentioned herein, the term “vector” or “recombinant vector” refers to a vector capable of transferring the polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of the polynucleotide of the present invention. A vector suitable for cloning is referred to as “cloning vector”. Such a cloning vector ordinarily contains a multiple cloning site containing a plurality of restriction sites. Presently, there are a number of vectors available for cloning a gene, which depend on a slight difference (for example, the types or sequence of a restriction enzyme of a multicloning site) from manufacturers. For example, typical sites and manufacturers thereof are described in Molecular Cloning (3^{rd} edition) Sambrook, J and Russell, D. W., Appendix 3 (Volume 3), Vectors and Bacterial strains. A3.2 (Cold Spring Harbor USA, 2001), and those skilled in the art may use such depending on the purpose of interest.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers. It is well known to those skilled in the art that the type of an organism (e.g., an animal) expression vector and the type of a regulatory element may vary depending on the host cell.

Expression vectors used herein include, for example, lambda FIX vector (phage vector) for screening a genomic library, lambda ZAP vector (phage vector) for screening cDNA. pBluescript II SK+/−, pGEM, pCR2.1 vectors (plasmid vectors) can be typically used for cloning a genomic DNA. pSV2neo vector (plasmid vector) may be used as an expression vector. Such vectors may be practiced in view of Molecular Cloning A3.2 supra.

As used herein, the term “terminator” refers to a sequence which is located downstream of the protein-encoding region of a gene and which is involved in the termination of transcription when DNA is transcribed into mRNA, and the addition of a poly-A sequence. It is known that a terminator contributes to the stability of mRNA, and has an influence on the gene expression levels.

As used herein, the term “promoter” refers to a base sequence which determines the initiation site of gene transcription and is a DNA region which directly regulates the frequency of transcription. Transcription is started by RNA polymerase binding to the promoter. Accordingly, a portion having promoter function of a gene herein refers to “promoter moiety”. The promoter region is usually located within about 2 kbp upstream of the first exon of a putative protein coding region. Therefore, it is possible to estimate a promoter region by predicting the protein coding region in a genomic base sequence using DNA analysis software. The putative promoter region is usually located upstream of a structural gene, but depending on the structural gene, i.e., downstream of a structural gene. Preferably, the putative promoter region is located within about 2 kbp upstream of the translation initiation site of the first exon.

As used herein, the term “enhancer” refers to a sequence which is used so as to enhance the expression efficiency of a gene of interest. Such enhancer is well known in the art.

One or more enhancers may be used, or no enhancer may be used.

As used herein, the term “operably linked” indicates that a desired sequence is located such that expression (operation) thereof is under control of a transcription and translation regulatory sequence (e.g., a promoter, an enhancer, and the like) or a translation regulatory sequence. In order for a promoter to be operably linked to a gene, typically, the promoter is located immediately upstream of the gene. A promoter is not necessarily adjacent to a structural gene.

As used herein the term “to process so as to transiently dysfunction” refers to the process in which a function of a certain gene is transiently made unfunctional, and includes for example, location of a gene so as to be unfunctional in the presence or absence of an agent (for example, a metal, antibiotic, and the like), which is responsible for on-off of a switch of the agent by operably linking a gene to another gene encoding the agent.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene Introduction & Expression Analysis”, Yodo-sha, 1997; and the like. Gene introduction can be confirmed by method as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like. Such cells are described in “Molecular Cloning (3^{rd} edition)” by Sambrook, J and Russell, D. W., Appendix 3 (Volume 3), Vectors and Bacterial strains. A3.2 (Cold Spring Harbor USA 2001).

Animal cells which can be used in genetic engineering or the like herein, include murine myeloma cells, rat myeloma cells, murine hybridoma cells, Chinese Hamster cells including CHO cells, BHK cells, African green monkey kidney cells, human leukemia cells, HBT5637 (see Japanese Laid-Open Publication 63-299), human colon cancer cell line and the like. Murine myeloma cells include ps20, NSO and the like; rat myeloma cells include YB2/0 and the like; human fetal kidney cells include HEK293 (ATCC: CRL-1573) and the like; human leukemia cells include BALL-1 and the like; African green monkey kidney cells include COS-1, COS-7 and the like; human colon cancer cell lines include HCT-15. Preferably, for example, cells include but are not limited to COS-1, NIH3T3, ES (R1, TMA, NR2) cells and the like.

Any method for introduction of DNA can be used herein as a method for introduction of a recombinant vector, including, for example, a calcium chloride method, an electroporation method (Methods. Enzymol., 194, 182 (1990)), a lipofection method, a spheroplast method (Proc. Natl. Acad. Sci. USA, 84, 1929 (1978)), a lithium acetate method (J. Bacteriol., 153, 163 (1983)), (Proc. Natl. Acad. Sci. USA, 84, 1929 (1978) and the like.

The transient expression of Cre enzyme, DNA mapping on a chromosome, and the like, which are used herein in methods for removing a genome, a gene locus, or the like, are well known in the art, as described in Kenichi Matsubara and Hiroshi Yoshikawa, editors, Saibo-Kogaku [Cell Engineering], special issue, Experiment Protocol Series “FISH Experiment Protocol From Human Genome Analysis to Chromosome/Gene diagnosis”, Shujun-sha (Tokyo), and the like.

Gene expression (e.g., mRNA expression, polypeptide expression) may be “detected” or “quantified” by an appropriate method, including mRNA measurement and immunological measurement methods. Examples of molecular biological measurement methods include Northern blotting, dot blotting, PCR, and the like. Examples of immunological measurement methods include ELISA, RIA, fluorescent antibodies, Western blotting, immunohistological staining, and the like, where a microtiter plate may be used. Examples of quantification methods include ELISA, RIA, and the like. Gene analysis methods using arrays (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat. Genet. 2002 December; 32 Suppl:526-32. Examples of methods for analyzing gene expression include, but are not limited to, RT-PCR, RACE, SSCP, immunoprecipitation, two-hybrid system, in vitro translation, and the like in addition to the above-described techniques. Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

### Screening

As used herein, the term “screening” refers to the selection of a target, such as an organism, a substance (for example, gene), or the like, or a given specific property of interest from a population containing a number of elements using a specific operation/evaluation method. For screening, the cell of the present invention may be used.

As used herein, screening by utilizing an immunological reaction is also referred to as “immunophenotyping”. Various techniques employ monoclonal antibodies to screen for a cell population expressing a marker. Examples of such techniques include, but are not limited to, magnetic separation using magnetic beads coated with antibodies, “panning” using antibodies attached to a solid matrix (i.e., a plate), flow cytometry, and the like (e.g., U.S. Pat. No. 5,985,660; and Morrison et al., Cell, 96:737-49 (1999)).

Screening may be performed using libraries obtained in vitro, in vivo, or the like (with a system using a real substance) or alternatively in silico (with a system using a computer). It will be understood that the present invention encompasses compounds having desired activity obtained by screening. The present invention is also intended to provide drugs, diagnostic agent and therapeutic agents which are produced by computer modeling based on the disclosures of the present invention.

As used herein the term “library” refers to a collection of genes, compounds, cells or the like for screening. Libraries may be a collection of genes, compounds, cells or the like having similar properties or random genes, compounds cells or the like. Preferably, collections of genes, compounds, cells or the like expected to have similar properties are used, but are not limited thereto.

Variants made according to the present invention (for example, ES cells), have been introduced to have modifications over the entire genome, and thus it is understood that the collection thereof may be used as a useful library for the analysis of a variety of genes.

It will also be understood that the patents, patent applications and literature cited herein should be incorporated by reference as if set forth fully herein as if the entirety were specifically described therein.

Hereinabove, the present invention has been described with preferable embodiments for ease of understanding. Hereinafter, the present invention will be described by way of examples. The examples below are provided only for illustrative purposes. Therefore, the scope of the present invention is limited only by the accompanying claims but not the examples.

### EXAMPLES

The animals used in the following examples have been cared for in accordance with the guidelines defined by Osaka University.

### Example 1

### Generation of Conditional Blm ES Cells

(Method for Generation of Conditional Blm ES cells)

Genomic DNA containing the mouse Blm gene (SEQ ID NOS: 1 and 2) was isolated from the R1-ES genomic library. The targeting vector was introduced into R1-ES cells and selected by using G418 and/or puromycin (Sigma). Targeted clones were screened by polymerase chain reaction (Expand High Fidelity PCR System (Roche) and confirmed by Southern blot analysis (Rapid-hyb buffer (Amersham-Pharmacia). All targeted clones used in this study possessed a normal karyotype.

### Western Blot Analysis

Blm^{tet/tet} ES cells were cultured with 1.0 μg/ml of doxycycline (dox; Sigma) and collected at appropriate time points. To examine Blm expression after dox withdrawal, cells cultured in dox-containing media were washed once with PBS and further cultured in the absence of dox until collection. Blm protein was detected with an ab476 antibody directed against BLM (Abcam). For Western blotting analysis, ECL Western Blotting Detection Reagents (Amersham-Pharmacia) were used.

### SCE Analysis

Blm^{tet/tet} ES cells were cultured in the presence or absence of dox for appropriate periods of time were labelled with 3 μg/ml of 5-bromodeoxyuridine (BrdU; Sigma) for 20 h and treated with 0.1 μg/ml of colcemid (KaryoMax-Colcemid (Invitrogen)) for 45 min. Chromosome spreads were stained with 0.1 mg/ml of acridine orange (Sigma).

### Functional Analysis

Targeting vector for the Fasl locus with the mutant neo gene (see, Koike et al., EMBO Rep. 3, 433-437 (2002); SEQ ID NOS: 4-5) was introduced into Blm^{tet/tet} ES cells. The rate of bi-allelic mutagenesis was measured by means of Luria-Delbruck functional analysis as described (see, Koike et al., EMBO Rep. 3, 433-437 (2002)). In brief, Blm^{tet/tet} ES cells were cultured for 24 h with or without 1.0 μg/ml of dox and plated on a 100-mm dish with a clonal-density culture with or without dox to obtain single-cell clones. Expanded single-cell clones were then selected by using high-dose G418 (1.0 mg/ml) without dox. The number of high-dose G418 resistant clones was counted 10 days after selection.

(ENU Mutagenesis and Screening for GPI-Anchor Mutants)

We carried out ENU mutagenesis and calculated the mutation frequency of the Hprt locus as described (Chen, Y. et al., Nature Genet. 24, 314-317 (2000)). The mutagenized ES cells were cultured with or without dox for 4 days to generate the mutant ES cell library. The mutant and control libraries were treated in 10 nM proaerolysin (Protox Biotech) in suspension (1.0×10⁶ cells per ml) and plated on a gelatin-coated 100-mm dish at 5-8×10⁶ cells per dish. The next day, dead cells were washed out and living cells were treated in 5 nM proaerolysin for 8 h before mitomycin-C (available from Kyowa Hakko KK). Treated feeder cells were added to generate GPI-anchor-defective mutant colonies. The resulting colonies were collected, expanded and transfected with GFP-GPI expression vector and cDNAs involved in GPI-anchor biosynthesis for the complementation assay (TransFast transfection reagent (Promega), DNA 2 mg and TransFast (12 μl) were added to 1.0×10⁵ ES cells).

### Results

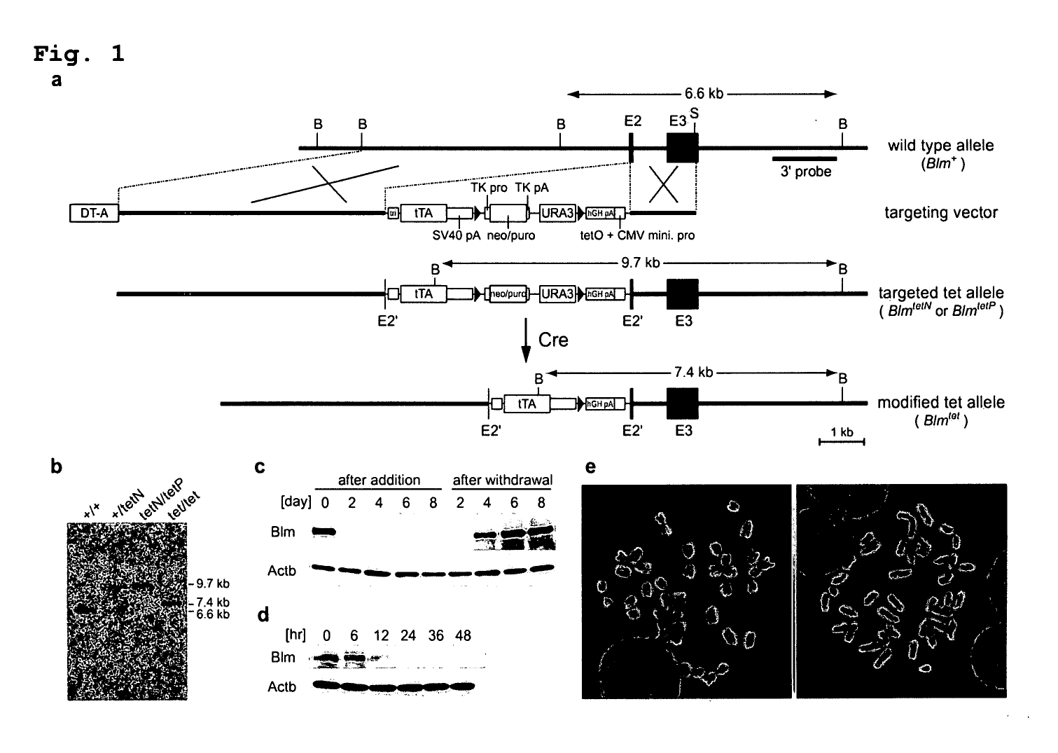

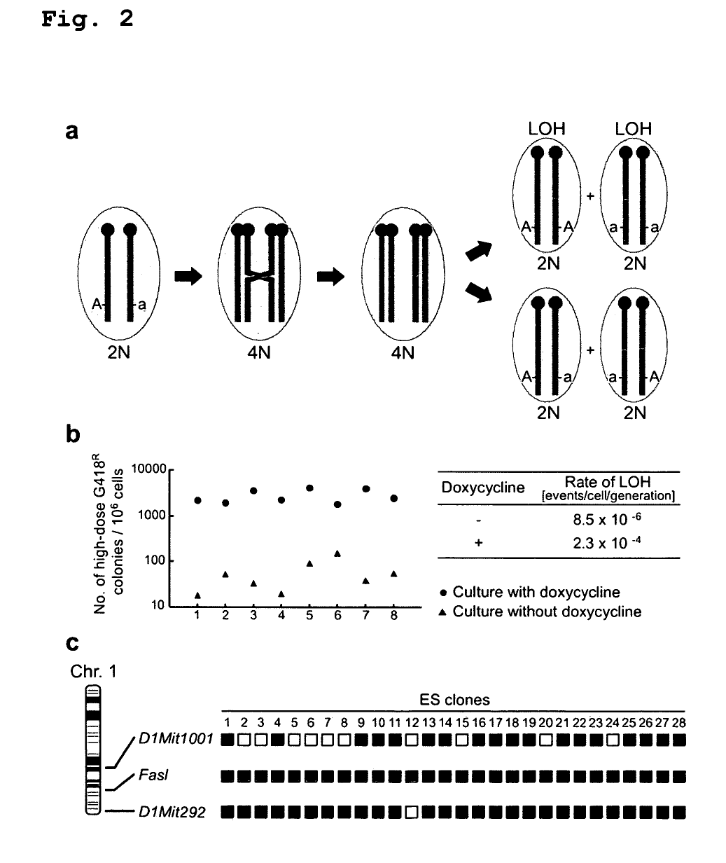

FIG. 1a shows general scheme of the Blm allele under conditional regulation of tetracycline (Blm^{tet}) (SEQ ID NO: 3). Tetracycline-system-based regulatory cassettes (tet cassettes) (see, Bond, C. T. et al., Science 289: 1942-1946 (2000)) were inserted immediately upstream of the translation initiation codons of both alleles of Blm to change them into Blm^{tet}. Targeting was confirmed by Southern blot analysis (FIG. 1b). We considered that continuous deficiency of Blm would cause continuous accumulation of bi-allelic mutations in the genome, resulting in a change in phenotype during long-term culture, while transient loss of Blm caused by Blm^{tet} would minimize changes in the phenotype. Regulation of Blm expression was examined by using the tetracycline analogue, doxycycline (dox). Addition of dox resulted in a rapid reduction in Blm protein (FIGS. 1c and 1d). Notably, the Blm protein regained its original expression after the withdrawal of dox (FIG. 1c). Increased numbers of sister chromatid exchanges (SCEs), a typical cytogenetic phenomenon of Bloom's syndrome cells (see, German, J., Dermatol. Clin. 13:7-18 (1995)), were observed (FIG. 1e), while Blm proteins were undetectable (FIG. 1c). SCE is closely coupled to homologous recombination in vertebrate cells (see, Sonoda, E. et al., Mol. Cell. Biol. 19; 5166-5169 (1999)); therefore, dox treatment is expected to induce recombination between homologous chromosomes and, when it occurs at the 4N stage, a mono-allelic mutation will become bi-allelic after cell division (FIG. 2a). To examine the effect of transient Blm deficiency on the rate of bi-allelic mutation, we introduced, by means of gene targeting, a mutant neo gene into the Fas ligand (Fasl) locus as a model of ‘mono-allelic mutation’. We have previously shown that duplication of the mutant neo gene at this locus, which represents ‘bi-allelic mutation’, can be selected by high doses of G418 (see, Koike, H. et al., EMBO Rep. 3: 433-437 (2002)). The rate of the duplication determined by Luria-Delbruck fluctuation analysis (see, Luria, S. E. and Delbruck, M., Genetics 28: 491-510 (1943)) was 8.5×10⁻⁶ events per cell per generation in the absence of dox, but increased to 2.3×10⁻⁴ events per cell per generation in the presence of dox (FIG. 2b). Therefore, transient loss of Blm causes a 27-fold increase in the rate of bi-allelic mutation (FIG. 2b). The rates obtained in this study are similar to those reported previously for loss of heterozygosity (LOH) in wildtype (2.3×10⁻⁵) and Blm-deficient (4.2×10⁻⁴) ES cells (see, Luo, G. et al., Nature Genet. 26: 424-429 (2000)).

To determine the chromosomal locations of the crossover, we used polymorphic markers in chromosome 1 of the R1-ES cell line (FIG. 2c), which was established from an F1 embryo obtained from the breeding of two different inbred 129 substrains (129×1/SvJ×129S1/SvImJ) (see, Lefebvre, L., Dionne, N., Karaskova, J., Squire, J. A. and Nagy, A., Nature Genet. 27: 257-258 (2001)). The respective locations of the D1Mit1001 and D1Mit292 markers are located roughly 30 megabases (Mb) proximal and distal from the Fasl locus. Twenty-eight high-dose G418-resistant clones were chosen to determine the chromosomal locations of the crossover, and ten (ca. 35%) crossovers were found to occur in a region 30-Mb proximal from the Fasl locus (FIG. 2c).

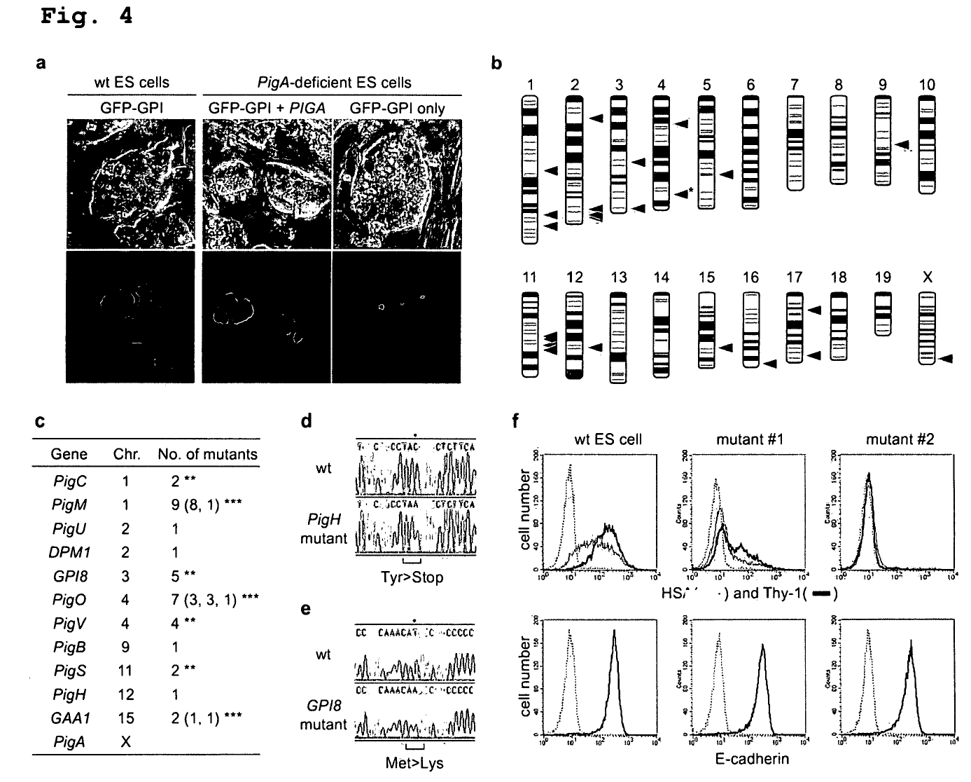

The observation of increased mitotic recombination in Blm-modified ES cells prompted us to examine the possibility of establishing an ES cell library containing the bi-allelic mutations throughout the genome. N-Ethyl-N-nitrosourea (ENU) was used as a mutagen because of its extremely high mutagenicity in ES cells (see, Chen, Y. et al., Nature Genet. 24: 314-317 (2000)). To determine whether the distribution and complexity of bi-allelic mutations were high enough to cover the whole genome, we screened for mutant ES cells deficient in glycosylphosphatidylinositol (GPI)-anchor biosynthesis. At least 23 genes, which are widely distributed in the mouse genome, are involved in this pathway (FIG. 4b). Because mutations in any gene in the GPI pathway yield a deficiency of GPI-anchored proteins on the cell surface, cells deficient in the GPI-anchor can be positively selected by using aerolysin, which kills cells with GPI-anchors (see, Hong, Y. et al., EMBO J. 21: 5047-5056 (2002)). The ES cell line used in this study was of male origin, whereas the PigA gene involved in the first step of GPI-anchor biosynthesis is localized on the X chromosome. Therefore, functional disruption of PigA does not require bi-allelic mutation and most of the GPI-anchor-deficient mutants would originate from PigA mutation (see, Kawagoe, K., Takeda, J., Endo, Y. and Kinoshita, T., Genomics 23: 566-574 (1994)). To avoid such a bias, extra copies of the PigA complementary DNA were introduced into the Blm^{tet/tet} ES cells before ENU mutagenesis.

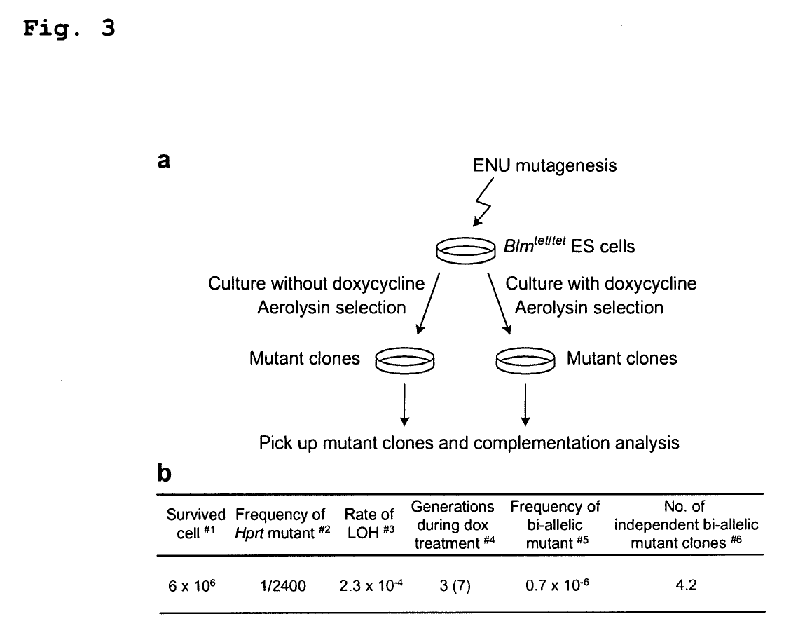

FIG. 3a summarizes the protocol for ENU mutagenesis of ES cells, generation of the ES cell library, and screening for GPI-anchor-deficient mutants. We treated 2×10⁸ ES cells using an ENU dose of 0.2 mg/ml for 2 h at 37° C. Cell viability was roughly 3%, resulting in 6×10⁶ cells surviving after ENU treatment. The mutation frequency in surviving cells was 1 in 2,400 at the X-linked monoallelic hypoxanthine phosphoribosyl transferase (Hprt) locus, as determined by selection with 6-thioguanine. It has been reported that ES cells treated at a higher concentration of ENU (0.35 mg/ml) retain germline competency (see, Chen, Y. et al., Nature Genet. 24: 314-317 (2000)), suggesting that the long-term viability of treated cells resembles that of wild-type ES cells. The frequency of bi-allelic mutations induced by dox treatment for three generations of cell cycles was calculated to be 0.7×10⁻⁶ per locus, and the number of independent clones bearing bi-allelic mutations was estimated at 4.2 per locus (FIG. 3b). These results indicate that our ES cell library contains bi-allelic mutations in most loci.

To verify the practical utility of this principle, we screened the ES cell library with aerolysin, which resulted in the isolation of 35 GPI-anchor-deficient mutants (FIG. 3a). When the GPI-anchored green fluorescent protein (GFP) construct (GFP-GPI) (see, Kondoh, G. et al., FEBS Lett. 458: 299-303 (1999)) was transfected into wild-type ES cells, GFP-GPI proteins were expressed on the cell surface (FIG. 4a). By contrast, GFP-GPI proteins were expressed on the mutants only when complementary cDNA was supplied (FIG. 4a). These mutants were therefore classified by means of complementation analysis using the transfection of cDNAs of genes involved in GPI-anchor biosynthesis. The mutated genes were found to be distributed widely throughout the genome, and mutants were identified in more than half of the known GPI-anchor biosynthesis genes (12 out of 23) after one round of screening (FIG. 4b) FIG. 4c shows that one mutant was obtained in four genes, but that more than one mutant was obtained in other autosomal genes. Because the genes that were affected frequently had same mutation, their mutants were probably derived from single clones. Therefore, the difference in mutant numbers would be explained by the differences in the stages at which the mutants were generated after the addition of dox. Sequence analysis of these mutants showed that they did not contain sequences of the wild-type allele (FIG. 4d, e), indicating that bi-allelic mutations had occurred in the ES cells treated with dox. Without dox treatment, only one mutant was isolated (data not shown). In addition, GPI-anchor biosynthesis was not complemented by cDNA transfection in two mutants, which suggested that these mutants had mutations in novel genes involved in the GPI pathway (FIG. 4f). Different genes were mutated in these mutants because the deficiency in GPI-anchor biosynthesis was complemented by cell fusion (data not shown).

We have shown that it is possible to isolate recessive mutants across the genome in mammalian cells. Although we (see, Koike, H. et al., EMBO Rep. 3: 433-437 (2002)) and others (see, Liu, P., Jenkins, N. A. and Copeland, N. G., Nature Genet. 30: 66-72 (2002) have reported a method to introduce bi-allelic mutation by Cre-loxP-mediated recombination between homologous chromosomes, that method can be applied only to a pre-selected chromosome that carries loxP sites on both alleles. Many, but not all, loci are functionally haploid in the Chinese hamster ovary (CHO) cell line (see, Gupta, R. S., Chan, D. Y. and Siminovitch, L., Cell 14: 1007-1013 (1978)), and the isolation of mutant cells with recessive phenotype has been reported (see, Hanada, K. et al., Nature 426: 803-809 (2003)). Because the probability of isolating mutants depends largely on whether target genes are functionally haploid or diploid in CHO cells, non-random isolation of mutants can be expected (see, Nakamura, N. et al., J. Biol. Chem. 272: 15834-15840 (1997)). In our study, more than half of the genes known to be involved in GPI-anchor biosynthesis could be identified in a single round of selection in ES cells with normal karyotype, thereby verifying the random nature of our selection scheme. Theoretically, bi-allelic mutations occur in most loci (FIG. 3b), but nearly half of the genes involved in GPI-anchor biosynthesis could not be identified. This incomplete coverage may be explained by AT base-pair predominant mutations with ENU in ES cells (see, Munroe, R. J. et al., Nature Genet. 24: 318-321 (2000)). This possibility can be tested by other chemical mutagens such as EMS and ICR191, which have a different mutation spectrum.

To identify genes responsible for a given phenotype, expression cloning can be applied. The development of highly efficient systems for cDNA library transduction and recovery, such as an episomal vector stably maintained in ES cells (see, Chambers, I. et al., Cell 113: 643-655 (2003)) and high-titer retroviral vectors resistant to promoter silencing in ES cells (see, Kitamura, T. et al., Exp. Hematol. 31: 1007-1014 (2003)), will greatly aid the identification of mutated genes. A survey of homologous regions by means of polymorphic markers is likely to narrow down the location of the mutation, as exemplified by FIG. 2c. To achieve fine mapping of mutations, we introduced Blm^{tet} alleles into C57BL/6×129S4/SvJae F1 hybrid ES cells (see, Eggan, K. et al., Proc. Natl. Acad. Sci. USA 98: 6209-6214 (2001)), for which a large number of polymorphic markers are available. Alternatively, tagged mutagenesis such as gene trap (see, Friedrich, G. and Soriano, P., Genes Dev. 5: 1513-1523 (1991)) may be used in place of ENU to facilitate identification of causative genes. In fact, the retroviral genetrap vector has been used successfully in the accompanying paper for a genome-wide recessive screen.

For phenotype-based genetic screening, it is essential to establish definitive criteria for judging whether an observed phenotype is caused by genetic mutations or by a simple change in the characteristics of wild-type cells. This is especially important in the analysis of ES cells, because a small fraction of the ES cell population may differentiate spontaneously even when culture conditions are carefully controlled. Conditional regulation of Blm expression is useful in assessing a given phenotype. If clones with the desired phenotype can be obtained more efficiently under dox-treated than under nonselective culture conditions, then those clones isolated by dox-induced LOH probably contain bi-allelic mutations. Here, the fact that 35 aerolysin-resistant clones were obtained from dox-treated culture, as compared with only 1 clone with non-selective culture, prompted us to characterize those clones further. Because pluripotent ES cells can differentiate into any type of tissue, a method for the comprehensive isolation of bi-allelic mutants should have a major impact on the analysis of molecular mechanism of differentiation in vitro as well as in vivo.

### LIST OF REFERENCES

1. German, J. Dermatol. Clin. 13, 7-18 (1995).
2. Groden, J., Nakamura, Y. & German, J. Proc. Natl. Acad. Sci. USA 87, 4315-4319 (1990).
3. Luo, G. et al. Nature Genet. 26, 424-429 (2000).
4. Kyba, M. & Daley, G. Q. Exp. Hematol. 31, 994-1006 (2003).
5. Kim, J. H. et al. Nature 418, 50-56 (2002).
6. Parisi, S. et al. J. Cell Biol. 163, 303-314 (2003).
7. Reubinoff, B. E., Pera, M. F., Fong, C. Y., Nature Biotechnol. 18, 399-404 (2000).
8. Thomson, J. A. et al. Science 282, 1145-1147 (1998).
9. Bond, C. T. et al. Science 289, 1942-1946 (2000).
10. Sonoda, E. et al. Mol. Cell. Biol. 19, 5166-5169 (1999).
11. Koike, H. et al. EMBO Rep. 3, 433-437 (2002).
12. Luria, S. E. & Delbruck, M. Genetics 28, 491-510 (1943).
13. Lefebvre, L., Dionne, N., Karaskova, J., Squire, J. A. & Nagy, A. Nature Genet. 27, 257-258 (2001).
14. Chen, Y. et al. Nature Genet. 24, 314-317 (2000).
15. Hong, Y. et al. EMBO J. 21, 5047-5056 (2002).
16. Kawagoe, K., Takeda, J., Endo, Y. & Kinoshita, T. Genomics 23, 566-574 (1994).
17. Kondoh, G. et al. FEBS Lett. 458, 299-303 (1999).
18. Liu, P., Jenkins, N. A. & Copeland, N. G. Nature Genet. 30, 66-72 (2002).
19. Gupta, R. S., Chan, D. Y. & Siminovitch, L. Cell 14, 1007-1013 (1978).
20. Hanada, K. et al. Nature 426, 803-809 (2003).
21. Nakamura, N. et al. J. Biol. Chem. 272, 15834-15840 (1997).
22. Munroe, R. J. et al. Nature Genet. 24, 318-321 (2000).
23. Chambers, I. et al. Cell 113, 643-655 (2003).
24. Kitamura, T. et al. Exp. Hematol. 31, 1007-1014 (2003).
25. Eggan, K. et al. Proc. Natl. Acad. Sci. USA 98, 6209-6214 (2001).
26. Friedrich, G. & Soriano, P. Genes Dev. 5, 1513-1523 (1991).

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

### INDUSTRIAL APPLICABILITY

In the present invention, mutations may be universally induced to stem cells such as ES cells, and the mutations may be introduced over the entire genome, and thus a stem cell library which may be used for analyzing the variety of genes is provided. This library may be used for developing pharmaceuticals, analyzing diseases, diagnosing diseases, therapy, gene therapy and the like, and thus highly industrially applicable.

What is claimed is:

1. A stem cell with a modification incorporated into both strands of the alleles thereof.

2. A stem cell according to claim 1, which is an embryonic stem cell.

3. A stem cell according to claim 1, in which the Bloom's syndrome (Blm) gene has been deleted or modified such that the Blm gene does not function.

4. A stem cell according to claim 3, wherein the Bloom's syndrome (Blm) gene comprises the sequence set forth in SEQ ID NO:1 or a variant thereof.

5. A library of stem cells with a modification incorporated into both strands of the allele thereof, wherein the stem cells included in the library have incorporated the modification over the entire genome thereof.

6. A library according to claim 5, wherein the stem cell is an embryonic stem cell.

7. A library according to claim 5, wherein the Bloom's syndrome (Blm) gene of the stem cell has been deleted or has been modified such that the Blm gene does not function.

8. A library according to claim 7, wherein the Bloom's syndrome gene comprises the sequence set forth in SEQ ID NO: 1 or a variant thereof.

9. A method for producing a stem cell with a modification incorporated into both strands of the alleles thereof, the method comprising the steps of:
A) providing a stem cell;
B) making the Bloom's syndrome gene in the stem cell unfunctional; and
C) inducing mutation in the stem cell.

10. A method according to claim 9, wherein the Bloom's syndrome gene is processed so as to be transiently dysfunctional.

11. A method according to claim 10, wherein the Bloom's syndrome gene is processed so as to be transiently dysfunctional in the presence of an agent.

12. A method according to claim 11, wherein the agent is selected from the group consisting of tetracycline, doxycyclin, estrogen derivatives and progesteron derivatives.

13. A method according to claim 9, wherein the induction of mutation is selected from the group consisting of exposure to a mutagen, use of a transposon gene, exposure to ultraviolet and exposure to radioactive rays.

14. A method according to claim 9, further comprising the step of inducing homologous recombination.

15. A method according to claim 14 further comprising the step of inducing homologous recombination in the 4N phase of the cell, thereby inducing cell division after the induction.

16. A method according to claim 9, wherein the stem cell is an embryonic stem cell.

17. A method according to claim 9, wherein the embryonic stem cell is a mammalian embryonic stem cell.

18. A stem cell obtained by the method according to claim 9.

19. A stem cell according to claim 18, which is an embryonic stem cell.

20. A tissue obtained by a stem cell which is obtained by the method according to claim 9.

21. A biological organism obtained by a stem cell which is obtained by the method according to claim 9.

22. A tissue obtained from a stem cell according to claim 1.

23. A biological organism obtained from a stem cell according to claim 1.

24. Use of Bloom's syndrome gene or a variant thereof for the mutation of a stem cell.

25. Use according to claim 24, wherein the Bloom's syndrome gene is disrupted or modified to be unfunctional in the stem cell.

26. Use according to claim 24, wherein the Bloom's syndrome gene comprises the sequence set forth in SEQ ID NO:1 or a variant thereof.

27. Use of Bloom's syndrome gene or a variant thereof for manufacturing a composition for mutating the stem cell.

28. Use according to claim 27, wherein the Blm gene has deleted Bloom's syndrome (Blm) gene or has been modified such that the Blm gene does not function.

29. Use according to claim 27, wherein the Bloom's syndrome gene comprises the sequence set forth in SEQ ID NO: 1 or a variant thereof.


SEQUENCE LISTING (5 sequences)

SEQ ID NO: 1 - DNA, 4251 bp, Mus musculus
  CDS (1)..(4251)
atggctgctgttcctctgaacaatctacaagaacaactacagcgacactcagccagaaaacttaataatcaacccagcctttcaaaaccaaaatctttaggttttacttttaaaaagaaaacatcagagggtgatgtgtctgtcactagtgtgtccgtagtaaaaacacctgcgttaagtgataaagatgtgaacgtgtctgaggccttttcattcactgagtctccactccacaaaccaaagcagcaggcaaagattgaaggcttctttaaacatttccctggaaggcagcaaagcaaggggacctgctctgagccgtcactgccggccacggtacagactgctcaggacactttgtgcactacccccaaaacccccactgcgaagaaactgcccgtggctgttttcaagaaattagaatttagttcttctgcagactccctcagtgactgggctgatatggatgactttgatatgtcagcatcagatgcgtttgcttcactggctaaaaatcctgccacaagagtaagcaccgctcagaaaatgaaaaagactaagagaaacttctttaaaccaccacctcgtaaagccaatgcagtaaagactgacttgactcctccctcccccgaatgcctgcaagtggatttaacgaaggaatcggaggaggaggaggaggaggaggaggaggcggagggggcggactgcctgagcagggatgtgatctgcattgacaatgattctgcttctgaagagctcacggagaaagacacgcaggaaagccagtctttgaaagctcacttgggagctgaaagaggtgacagtgaaaagaagagccatgaagacgaagctgtgttccattcagttcagaacactgaatactttgaacacaatgacaatgattatgatatagattttgttccaccttctccagaagaaatcatctccactgcttcttcctcgttgaaatgttccagtatgttaaaggatcttgatgactctgacaaagaaaagggcattcttagcacctcagaagagcttctgtcaaaaccagaggaaatgaccacacacaagtctgatgcaggaaccagtaaagactgtgatgcccagcagatacgcatacagcagcagcttattcatgtgatggagcacatctgtaagttagttgatactgttcctactgatgaactggaagctttgaattgtgggaccgaattgcttcaacaacgaaacataaggaggaagctcctagctgaagcaggttttaatggaaatgacgtcagacttctgggttctctgtggaggcacaggcctgattcacttgataacacagtgcagggcgactcctgccctgtggggcatcctaataaagagttaaattctccataccttctctcacattccccttccactgaggaatgtttacccaccaccactccaggaaagacaggattctcagccaccccgaagaatctctttgaaaggccgttattgaattcccatttacagaagtcctttgtaagtagcaactgggctgaaacaccaaggatggaaaacaggaacgaaagcactgacttcccagggagtgttctcaccagcaccactgtgaaagctcagagtaaacaagctgcttcaggatggaacgtagagagacacggccaggcttcctatgatatcgataactttaatattgatgactttgatgatgatgatgatgatgatgactgggaaaacataatgcacaattttccagccagcaaatcttccacagccacctacccacccatcaaggaaggtgggccagttaaatctctctcagaaaggatttcttcagccaaggcaaagtttcttccagtggtatcaaccgctcaaaatacaaacctctcagagtcaattcagaattgctctgataagctggcccaaaatttatcatcaaaaaatccaaaacatgaacattttcaaagtcttaattttcctcatacaaaagaaatgatgaagattttccataagaaatttggcttgcataattttagaactaatcagctagaggcgatcaatgctgcgctgcttggtgaagactgctttatcctaatgcccactggaggaggtaaaagtttgtgctaccagctccctgcctgtgtttctcctggggtcacaattgtcatttctcccttgagatcactaatagtagatcaagtccaaaagctgacttcctttgatattccagctacatatctgacaggggataagactgactcagaagctgcaaatatttacctccaattatccaaaaaagacccaatcatcaagcttttgtatgttactccagagaaggtctgtgcaagtaacaggctgatttctactctggagaatctgtatgagcggaagctcttggcacgttttgtcattgatgaagcgcattgtgtgagtcagtggggtcatgattttcgtcaagattacaaaaggatgaatatgcttcgccagaagtttccttctgttccagtgatggccctcacggccacagcgaaccccagggtccagaaggacatcctcactcagctgaagatcctcagacctcaggtgtttagcatgagctttaacagacacaatctgaagtactatgtattacccaagaagcccaaaaaagtagcatttgattgcctagagtggatcagaaagcatcacccttatgactcggggataatttactgcctctccaggagggaatgtgacacaatggctgacactttacagagagaaggcctggctgccctggcttaccatgcgggcctcagtgactctgccagagatgaggtgcagcacaagtggatcaaccaggacaactgccaggttatctgtgcgacaattgcgtttggaatgggaattgacaaacctgacgtgcgatttgtgattcatgcatctcttcctaaatctatggagggttattatcaagaatccggccgagctggaagagatggggaaatatctcactgcgtgcttttctatacatatcatgatgtgaccagactgaagagacttataatgatggaaaaagatggaaactatcatacgaaggaaactcacgtcaataatctatatagcatggtacattactgtgaaaacataacggaatgcagaagaatacagcttttagcttactttggtgaaaaaggattcaaccctgatttttgtaagaaatacccagatgtttcttgtgacaattgctgtaaaacaaaggattataaaacaaaagatgtgactgatgacgtgaaaaatattataagatttgttcaagaacacagttcatcaccaggaacaagaaatataggacctgctggaagatttactctgaacatgctggtcgacattttcttggggagcaagagtgcaaaagttaagtctggaatatttggaaaggggactacatattcacgacataatgccgaaagactttttaaaaagctgattctagacaaaatcctggatgaagacttatatatcaatgccaatgaccaaccaattgcctatgtgatgctaggaacaaaagcccacagtgtgctgagtggccacttgaaggtggacttcatggaaacggaaaattccagcagtattaaaaaacaaaaagctttagtggccaaagtatcccagagagaagaggtagttaagaaatgtcttggagaacttacagaggtctgcaaattgctggggaaagtctttggtgtccattacttcaatatttttaatacagccacactcaaaaagcttgcagaatctttatcttctgatcctgaggttttgcttcagattgatggtgttaccgaagacaagctggaaaaatatggtgcagaagtgattccagtattacagaagtactcagaatggacagtgccagctgaggatggttccccaggcgccagaggcgccccagaggacactgaggaggaggaggaggaagcgcctgtatcttctcactactttgcaaatcaaactagaaatgaaagaaagagaaagaaaatgtcagccacccataagcccaagaggagaagaactagttacggtggcttcagagcaaaggggggctctactacatgcagaaaaacgacttctaaaagtaaattctatggtgtaactggatcccgctcggcctcatgtgcttctcaggcaacatcatcagccagtagaaaactggggattatggctcctccaaagcctgtaaatagaacgttcctcaggccttcatatgccttctcctaa

SEQ ID NO: 2 - protein, 1416 aa, Mus musculus
MAAVPLNNLQEQLQRHSARKLNNQPSLSKPKSLGFTFKKKTSEGDVSVTSVSVVKTPALSDKDVNVSEAFSFTESPLHKPKQQAKIEGFFKHFPGRQQSKGTCSEPSLPATVQTAQDTLCTTPKTPTAKKLPVAVFKKLEFSSSADSLSDWADMDDFDMSASDAFASLAKNPATRVSTAQKMKKTKRNFFKPPPRKANAVKTDLTPPSPECLQVDLTKESEEEEEEEEEAEGADCLSRDVICIDNDSASEELTEKDTQESQSLKAHLGAERGDSEKKSHEDEAVFHSVQNTEYFEHNDNDYDIDFVPPSPEEIISTASSSLKCSSMLKDLDDSDKEKGILSTSEELLSKPEEMTTHKSDAGTSKDCDAQQIRIQQQLIHVMEHICKLVDTVPTDELEALNCGTELLQQRNIRRKLLAEAGFNGNDVRLLGSLWRHRPDSLDNTVQGDSCPVGHPNKELNSPYLLSHSPSTEECLPTTTPGKTGFSATPKNLFERPLLNSHLQKSFVSSNWAETPRMENRNESTDFPGSVLTSTTVKAQSKQAASGWNVERHGQASYDIDNFNIDDFDDDDDDDDWENIMHNFPASKSSTATYPPIKEGGPVKSLSERISSAKAKFLPVVSTAQNTNLSESIQNCSDKLAQNLSSKNPKHEHFQSLNFPHTKEMMKIFHKKFGLHNFRTNQLEAINAALLGEDCFILMPTGGGKSLCYQLPACVSPGVTIVISPLRSLIVDQVQKLTSFDIPATYLTGDKTDSEAANIYLQLSKKDPIIKLLYVTPEKVCASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQREGLAALAYHAGLSDSARDEVQHKWINQDNCQVICATIAFGMGIDKPDVRFVIHASLPKSMEGYYQESGRAGRDGEISHCVLFYTYHDVTRLKRLIMMEKDGNYHTKETHVNNLYSMVHYCENITECRRIQLLAYFGEKGFNPDFCKKYPDVSCDNCCKTKDYKTKDVTDDVKNIIRFVQEHSSSPGTRNIGPAGRFTLNMLVDIFLGSKSAKVKSGIFGKGTTYSRHNAERLFKKLILDKILDEDLYINANDQPIAYVMLGTKAHSVLSGHLKVDFMETENSSSIKKQKALVAKVSQREEVVKKCLGELTEVCKLLGKVFGVHYFNIFNTATLKKLAESLSSDPEVLLQIDGVTEDKLEKYGAEVIPVLQKYSEWTVPAEDGSPGARGAPEDTEEEEEEAPVSSHYFANQTRNERKRKKMSATHKPKRRRTSYGGFRAKGGSTTCRKTTSKSKFYGVTGSRSASCASQATSSASRKLGIMAPPKPVNRTFLRPSYAFS

SEQ ID NO: 3 - DNA, 5315 bp, Artificial
  tet-off cassette sequence
ccaagctggccatactggcctgactagttctagaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcagcccaagcggatcctgcggataaccaccatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttgggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtcctggccggaccacgctatctgtgcaaggtccccggccccggacgcgcgctccatgagcagagcgcccgccgccgaggcgaagactcgggcggcgccctgcccgtcccaccaggtcaacaggcggtaacgtaaccggcctcttcatcgggaatgcgcgcgaccttcagcatcgccggcatgtccccctggcggacgggaagtatccagctcgaccaagcttctcgaggaattccgatcatattcaataacccttaatataacttcgtataatgtatgctatacgaagttattaggtctgaagaggagtttacgtccagccaagctagcttggctgcaggtcgagcagtgtggttttcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcacatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggccaataaaaagacagaataaaacgcacgggtgttgggtcgtttgttcggatcggccgcgtatcacgaggccagcttttcaattcatcattttttttttattcttttttttgatttcggtttccttgaaatttttttgattcggtaatctccgaacagaaggaagaacgaaggaaggagcacagacttagattggtatatatacgcatatgtagtgttgaagaaacatgaaattgcccagctattcttaacccaactgcacagtaacaaaaacatgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgcggccgatccgtcgaggaattccgatcatattcaataacccttaatataacttcgtataatgtatgctatacgaagttattaggtctgaagaggagtttacgtccagccaagctagcttggctgcaggtcgagtaccccgggttcgaaatcgatagatctcccgggtggcatccctgtgacccctccccagtgcctctcctggccctggaagttgccactccagtgcccaccagccttgtcctaataaaattaagttgcatcattttgtctgactagggtgtccttctataatattatggggtggagggggggtggtatggagcaaggggcaagttgggaagacacacctgtagggcctgcggggtctattgggaaccaagcttggagtgcagtggcacaatcttggctcactgcaatctccgcctcctgggttcaagcgattctcctgcctcagcctcccgagttgttgggatttccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatggcccaaccggtgggacatttgagttgcttgcttggcactgtcctctcatgcgttgggtccactcagtagatgcctgttgaattcggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattcctgcacttaagcctaggcggccgc

SEQ ID NO: 4 - DNA, 804 bp, Artificial
  mutant neo gene
atgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 5 - protein, 267 aa, Artificial
  Synthetic Construct
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF